(19) United States
(12) Patent Application Publication
(10) Pub. No.: US 2025/0170405 A1
(43) Pub. Date: May 29, 2025

(54) ELECTRICAL NEUROSTIMULATION SYSTEM

(71) Applicant: B. Braun Melsungen AG, Melsungen (DE)

(72) Inventor: Jens Wildhagen, Hannover (DE)

(21) Appl. No.: 18/956,246

(22) Filed: Nov. 22, 2024

(30) Foreign Application Priority Data
Nov. 24, 2023 (DE) 10 2023 132 855.7

(51) Int. Cl. A61N 1/36; A61N 1/05

(52) U.S. Cl. CPC A61N 1/36185; A61N 1/05; A61N 1/3614

(57) ABSTRACT

An electrical neurostimulation system, which can be used in pain therapy, includes a stimulator having a shaft and a distal end having a plurality of electrodes arranged along an axis of the shaft and configured for outputting electrical stimuli to a body tissue surrounding the shaft. A control device is connected to the electrodes for controlling the output of the electrical stimuli. The electrodes can be activated and deactivated independently of one another by the control device and activated for forming different electrode activation patterns along the axis. A determining device can determine an axial dislocation of the shaft. The control device is connected to the determining device and can axially move the electrode activation pattern by a change of the activation and deactivation of the electrodes as a function of the axial dislocation, to locally adapt output of the electrical stimuli to the axial dislocation of the shaft.

### CROSS-REFERENCE TO RELATED APPLICATION

This application claims priority under 35 U.S.C. § 119 to German Application No. 10 2023 132 855.7, filed on Nov. 24, 2023, the content of which is incorporated by reference herein in its entirety.

### FIELD

The present disclosure relates to a system for electrical neurostimulation, comprising a stimulator and a control device. The stimulator has an elongated stimulator shaft which has a distal stimulator end having a plurality of electrodes. The plurality of electrodes are arranged adjacently along a longitudinal axis of the stimulator shaft and configured for outputting electrical stimuli to a body tissue surrounding the stimulator shaft. The control device is connected to the plurality of electrodes and configured for controlling the output of the electrical stimuli. The plurality of electrodes can be activated and deactivated independently of one another by means of the control device and can thus be activated for forming different electrode activation patterns along the longitudinal axis.

### BACKGROUND

Systems of this type are generally known in the field of medical engineering and provided for use in a pain therapy. In a pain therapy termed peripheral nerve block (PNB), the stimulator is pushed so far distally that the distal stimulator end is positioned close to the nerve to be treated. After the positioning of the stimulator shaft, the electrodes which achieve an optimum stimulation result when forming a particular electrode activation pattern are selected for activation. An optimum stimulation result may for example be characterized by maximum pain reduction. The electrode activation pattern can for example be selected on the basis of patient feedback. A system of this type is known for example from U.S. Pat. No. 8,005,538 B2.

### SUMMARY

It is an object of the present disclosure to provide a system of the type mentioned in the introduction, which allows an improved pain therapy.

This object is achieved in that a determining device is present and configured for determining an axial dislocation of the stimulator shaft, wherein the control device is connected to the determining device and configured for axially moving the electrode activation pattern by means of a change of the activation and deactivation of the electrodes as a function of the determined axial dislocation, in order to locally adapt the output of the electrical stimuli to the axial dislocation of the stimulator shaft. The present disclosure is based on the consideration that inadvertent axial movements of the stimulator shaft can impair the effectiveness of the pain therapy. An inadvertent proximal pulling out or distal pushing in of the stimulator shaft leads to a change of the position of the electrodes in relation to the nerve to be stimulated. An initially selected electrode activation pattern can, if an axial dislocation occurs, lead to an only suboptimal stimulation of the nerve and as a result impair the effectiveness of the pain therapy. In order to counteract this, the control device moves the electrode activation pattern along the longitudinal axis of the stimulator shaft if an axial dislocation occurs. The electrode activation pattern is made to follow the axial dislocation that is determined, as it were. This ensures that, even after a possible dislocation of the stimulator shaft, the electrode activation pattern assumes the original position again in relation to the surrounding body tissue and in particular the nerve to be stimulated or maintains the original position in spite of the dislocation or a deviation of the position assumed after the dislocation from the original position is at least minimized. Thus, proximal dislocations of the distal stimulator end are compensated or their impairments are at least reduced by the solution according to the present disclosure. As a result, an always optimum or quasi-optimum stimulation of the surrounding body tissue or the nerve to be treated is ensured, which is robust with respect to external influences or movements of the patient. The movement of the electrode activation pattern to adapt to the axial dislocation takes place in particular by a length which corresponds to the distance of adjacent electrodes or a multiple of the same. In the case of a proximal dislocation of the stimulator shaft, the electrode activation pattern is moved distally. In the case of a distal dislocation of the stimulator shaft, the electrode activation pattern is moved proximally. A deviation of the newly assumed position from the original position is not important if, in spite of the deviation, the desired effect, particularly a pain-reducing effect, of the electrical stimulus on the nerve or the surrounding body tissue is achieved. Stimulus means any type of current that is output by means of the electrodes and/or voltage applied at the electrodes, in particular one individual or a plurality of successive current pulses or voltage pulses or a sustained output of current or a sustained application of a voltage. The more closely adjacent the electrodes are arranged along the longitudinal axis and the larger the number of electrodes is, the smaller can be a deviation which the position of the electrode activation pattern can have at most after the adaptation to the dislocation relative to the position before the dislocation. The deviation is particularly at most half of the distance between two electrodes. Preferably, the stimulator has a multiplicity of electrodes, preferably between 5 and 15 electrodes, but at least three electrodes. Advantageously, the electrodes assume one of two states. In this case, the electrode activation pattern that is formed from the sequence of activated and deactivated electrodes along the longitudinal axis of the stimulator shaft can be represented as binary code. For example, to output an electrical stimulus, two electrodes in the activated state can between them output a sequence of a positive current or voltage pulse and a negative current or voltage pulse, or vice versa, (what is known as a biphase signal or bipolar pulse signal). An electrode in the deactivated state then correspondingly outputs no signal. Alternatively to this, to output an electrical stimulus, an electrode in the activated state can output an AC voltage pulse or an alternating-current pulse, whilst the electrode in the deactivated state outputs no signal. The determining device is configured for determining the axial dislocation of the stimulator shaft. In principle, any measurement principle suitable for detecting length or position changes can be used for this, for example a capacitive, inductive or resistive measurement principle. In one embodiment, the determining device has at least one sensor which is arranged on or in the body of the patient or in the vicinity of the body and generates a signal representing the axial dislocation. In a further embodiment, the stimulator shaft is inserted into the body of the patient with the aid of an insertion aid and positioned close to the nerve that is to be treated. The insertion aid used can be a cannula and/or a capillary tube, wherein it is possible in principle to differentiate between different set-up techniques (“cannula over the needle”, “cannula through the needle”, “cannula through the capillary tube”). The stimulator can be termed a catheter or modulation catheter. The process of moving or adapting or compensating the electrode activation pattern can also be termed regulation or tracking. The movement of the electrode activation pattern can also be termed virtual movement.

In one embodiment of the present disclosure, the determining device is connected to the plurality of electrodes and configured for determining impedances between the electrodes and therefore an impedance pattern of the surrounding body tissue along the longitudinal axis of the stimulator shaft, for determining a temporal change of the impedance pattern and for determining the axial dislocation depending on the determined temporal change of the impedance pattern. Preferably, the determining device is configured for determining impedances between respectively adjacent electrodes. Alternatively or additionally, the determining device is configured for determining impedances between at least one of the electrodes and a skin electrode. The stimulator, more precisely: the plurality of electrodes, function in this embodiment as a sensor, as it were, for generating a signal representing the axial dislocation, so that no separate sensor is necessary for determining the dislocation. This constitutes a particularly advantageous embodiment of the determining device. This embodiment is based on the discovery that the impedance is dependent on specific properties of the surrounding body tissue and changes along the longitudinal axis of the stimulator shaft depending on the surrounding body tissue, i.e. an impedance curve or else an impedance pattern forms along the longitudinal axis. Impedance means the complex electrical resistance of the surrounding body tissue. The determining device approximates the impedance curve along the stimulator shaft by means of the impedance pattern that is determined. In the event of an axial dislocation of the stimulator shaft, the impedance curve is maintained, as it is a property of the body tissue surrounding the stimulator shaft. The impedance curve moves however in relation to the stimulator shaft, more precisely: the electrodes arranged on the stimulator shaft, along its longitudinal axis counter to the direction of the dislocation. To determine the axial dislocation, two impedance patterns that were determined one after the other temporally are preferably compared with one another and the axial dislocation is determined depending on a comparison result. Determination is preferably understood as a measurement or detection. In one embodiment, the impedances are determined continuously or at regular or irregular time intervals. The faster a temporal change of the impedance pattern is determined, the faster the system or the control device can react to it and adapt the electrode activation pattern to the axial dislocation. In a further embodiment, the movement of the electrode activation pattern takes place as soon as a temporal change of the impedance pattern is determined, which exceeds a predetermined limit. In a further embodiment, a resolution of the impedance curve along the longitudinal axis corresponds to a distance of adjacent electrodes. In an alternative embodiment, impedances are determined between non-adjacent electrodes, e.g. between a first electrode and a next-but-one electrode, as a result of which a higher resolution of the impedance pattern may be possible, and/or impedances are determined between the electrodes and a further separate, particularly large-area, base electrode, which can be configured as a skin electrode for application onto the skin of the patient. In a further embodiment, the frequency with which the determining device determines an impedance pattern can be changed and in particular can be adapted to external circumstances. For example, this frequency, i.e. the time interval of two successively determined impedance patterns, can be reduced if the patient will likely move and the risk of a dislocation increases as a result.

In a further embodiment of the present disclosure, the determining device is configured for determining the temporal change of an impedance spectrum of the impedances.

In a further embodiment of the present disclosure, the determining device is configured for determining the temporal change of the impedance pattern, taking account of a real part and/or an imaginary part of the impedances that are determined. In a further embodiment of the present disclosure, the determining device is configured for determining the temporal change of the impedance pattern, taking account of a magnitude and/or a phase of the impedances that are determined. In a further embodiment of the present disclosure, the determining device is configured for determining the temporal change of the impedance pattern, taking account of a spectrum of the impedances that are determined. Thereby, in each case an accurate representation of the impedance pattern or an approximation of the impedance curve and as a result an accurate and reliable determination of the axial dislocation is possible. The spectrum of the impedances that are determined can be a frequency and/or amplitude spectrum. Such an analysis of the impedance pattern can be termed impedance spectroscopy. In one embodiment, a plurality of the properties real part, imaginary part, magnitude, phase and spectrum of the impedances are taken into account together for determining the temporal change of the impedance pattern. The determining device is preferably configured for determining a/the temporal change of the impedance spectrum.

In a further embodiment of the present disclosure, the plurality of electrodes are configured for outputting a measuring current for the determination of the impedances. In addition, the control device is configured for controlling the output of the measuring current. As a result, no further electrodes and no further separate device is necessary for generating the measuring current. In addition, possible interactions of the measuring current with the surrounding body tissue can be limited to a local region around the distal stimulator end, as a result of which the surrounding body tissue is treated with care. In addition, small measuring currents can be used owing to the comparatively small distances between the electrodes. In one embodiment, the output of the measuring current takes place continuously in the form of a direct current or alternating current. In a further embodiment, the output of the measuring current takes place discretely as pulses at regular or irregular intervals.

In a further embodiment of the present disclosure, the measuring current has a current intensity of at most 10 mA, preferably at most 0.5 mA, particularly preferably at most 0.01 mA. Due to the low current intensity of the measuring current, effects on the surrounding body tissue and interactions with the surrounding body tissue can be avoided or at least kept small.

In a further embodiment of the present disclosure, the measuring current has an alternating current which has a frequency of at least 10 Hz, preferably at least 1 kHz. The use of alternating current enables the determination of a real impedance, i.e. an impedance for which the imaginary part is not zero. As a result, a temporal change of the impedance can be determined more accurately and more reliably than in the case of the use of direct current. The use of a high frequency of the alternating current can reduce a risk of stimulation of motor nerves.

In a further embodiment of the present disclosure, the measuring current is an alternating current which has a frequency spectrum, particularly a sweep. The use of a plurality of frequencies enables a more accurate and/or more robust measurement result. Frequency spectrum means that the alternating current has a plurality of frequencies and the measuring current therefore represents a multifrequency signal. For example, the measuring current can have a plurality of frequency components with 10 Hz, 1 kHz, 10 kHz and 1 MHz. In this case, the individual frequency components can arise simultaneously and/or one after the other. In signal processing, one understands a sweep to mean an alternating current, the frequency of which continuously passes through a predetermined range. The range is preferably defined by a minimum and a maximum frequency.

In a further embodiment of the present disclosure, the determining device is configured for determining impedances on the basis of the electrical stimuli that are output. In one embodiment, the determining device is configured for determining impedances across a first group of electrodes when an electrical stimulus is output by a second group of electrodes, wherein, in particular, the electrodes of the first group are not contained in the second group. For example, two electrodes in each case or by interacting with one another output an electrical stimulus which is used as a measurement signal for determining the impedances. Specifically, this measurement signal can then be used by the remaining electrodes, which are not used for outputting the stimulus, for determining impedances. In a further embodiment, the determining device is configured for determining impedances both on the basis of an electrical stimulus that is output and on the basis of a separate measuring current.

The present disclosure additionally relates to a method for operating a system according to the preceding disclosure. The method according to the present disclosure is provided for operating a system for electrical neurostimulation, wherein the system has a stimulator having an elongated stimulator shaft, which has a distal stimulator end having a plurality of electrodes. The method comprises the following steps: activating the plurality of electrodes, forming an electrode activation pattern with activated electrodes and non-activated, i.e. deactivated, electrodes; determining an axial dislocation of the stimulator shaft; activating the plurality of electrodes, wherein the electrode activation pattern is moved axially depending on the determined axial dislocation by means of a changed activation and deactivation of the electrodes, in order to compensate the axial dislocation of the stimulator shaft. The method according to the present disclosure enables the same advantages as the system according to the present disclosure, so it is possible to have reference to the corresponding embodiments. The disclosure applying to the system according to the present disclosure and its embodiments also applies mutatis mutandis for the method according to the present disclosure and embodiments of the same.

### BRIEF DESCRIPTION OF THE DRAWINGS

Further advantages and features of the present disclosure can be found in the following description of preferred exemplary embodiments of the present disclosure, which are presented with reference to the drawings.

FIG. 1 shows an embodiment of a system according to the present disclosure, in a schematic side view, for electrical neurostimulation, the system comprising a stimulator which has a stimulator shaft having a plurality of electrodes, a control device and a determining device.

FIG. 2 by way of example shows an impedance curve of a body tissue surrounding the stimulator shaft along a longitudinal axis of the stimulator shaft.

FIG. 3 shows, in a schematic side view, the stimulator with an electrode activation pattern and a determined impedance pattern before the occurrence of an axial dislocation.

FIG. 4 shows, in a schematic side view, the stimulator with a moved electrode activation pattern after the occurrence of the axial dislocation.

### DETAILED DESCRIPTION

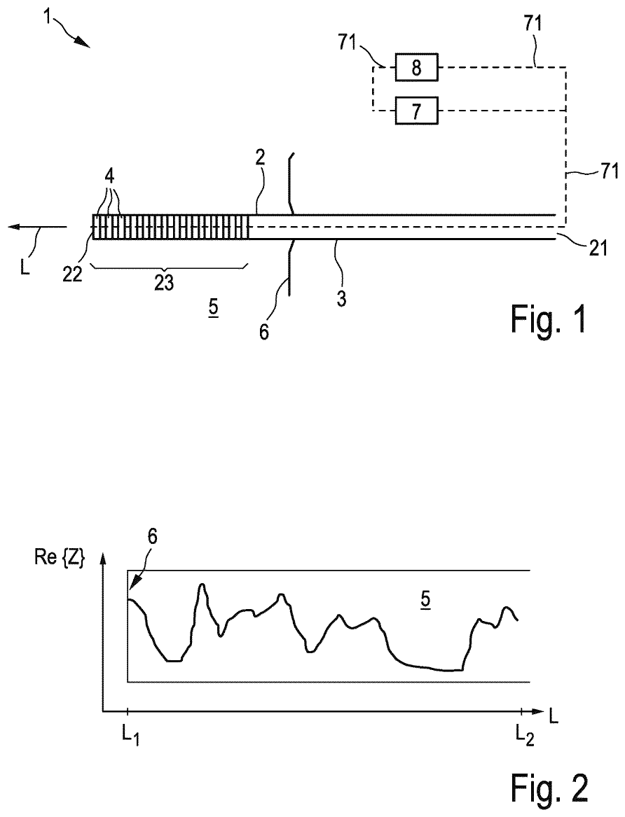

According to FIG. 1, a system 1 for electrical neurostimulation is provided. The system 1 has a stimulator 2.

In the embodiment shown, the stimulator 2 is formed as a catheter and is used specifically as a pain catheter in a pain therapy which is also termed peripheral nerve block.

The stimulator 2 has a stimulator shaft 3 and a plurality of electrodes 4. The stimulator shaft 3 is elongated along a longitudinal axis L between a proximal stimulator end 21 and a distal stimulator end 22.

The electrodes 4 are arranged adjacently to one another along the longitudinal axis L of the stimulator shaft 3 and in the region of the distal stimulator end 22. The said region is also termed the distal end section 23 in the following.

In the use of the system 1, the stimulator 2 is positioned, with the distal end section 23 at the front, in the body tissue 5 of a patient in the direct vicinity of a nerve that is to be anaesthetized. To set up the stimulator 2, the stimulator shaft 3 is positioned in the body tissue 5 possibly with the aid of an insertion aid. Electrical stimuli can be output to the nerve to be anaesthetized by means of the electrodes 4. The stimuli are used for pain reduction.

The system 1 further comprises a control device 7 which is connected to the electrodes 4 by means of an electrical signal line 71. The control device 7 is configured to activate and to deactivate the electrodes 4 independently of one another and via the signal line 71. The signal line 71 preferably runs along and in the interior of the stimulator shaft 3. The electrodes 4 in each case assume one of two states (activated/deactivated) and as a result form an electrode activation pattern 41 along the longitudinal axis L. The electrical stimulus output by means of the electrodes 4 to the body tissue 5 or the nerve is characteristic for the electrode activation pattern 41. By corresponding activation of the electrodes 4, the control device 7 thus controls the electrical stimulus that is output to the body tissue 5 or the nerve.

The system 1 further has a determining device 8. In the present case, this is connected by means of the electrical signal line 71 to the control device 7. In addition, the determining device 8 is connected by means of the signal line 71 to the electrodes 4.

The determining device 8 is configured for determining impedances Z between respectively adjacently arranged electrodes 4, wherein the impedances Z—in the applied state of the stimulator 2—are determined substantially by the body tissue 5 surrounding the distal end section 23. To determine the impedances Z, the control device 7 activates the electrodes 4 to output a measuring current. The measuring current can be a wideband signal, particularly a signal consisting of a plurality of frequencies or a sweep, for example having a maximum current intensity of e.g. 10 mA and a frequency range between 10 Hz and 1 MHz. In order to avoid undesired nerve reactions, the frequency range can be limited to high-frequency signals above e.g. 1 kHz. Alternatively, a wideband pulse signal can be used as measuring current. In one embodiment, an electrical stimulus that is output by means of the electrodes 4 can be used as measurement signal for determining impedances Z, as long as the electrical stimulus is suitable for this, for example is configured as a pulse signal.

The impedance Z changes over the body tissue 5, depending on the local properties of the body tissue 5. FIG. 2 shows an exemplary impedance curve Z along the longitudinal axis L in the body of a patient. The point L1 on the abscissa which represents the longitudinal axis L corresponds in the present case to an entry point of the stimulator shaft 3 into the body tissue 5. From point L1 onwards along the longitudinal axis L up to a point L2, the impedance Z changes continuously depending on the location along the longitudinal axis L, represented in FIG. 2 by the real part Re{Z} of the impedance. It is possible to see that different sections along the longitudinal axis L differ with regards to the impedance Z determined there.

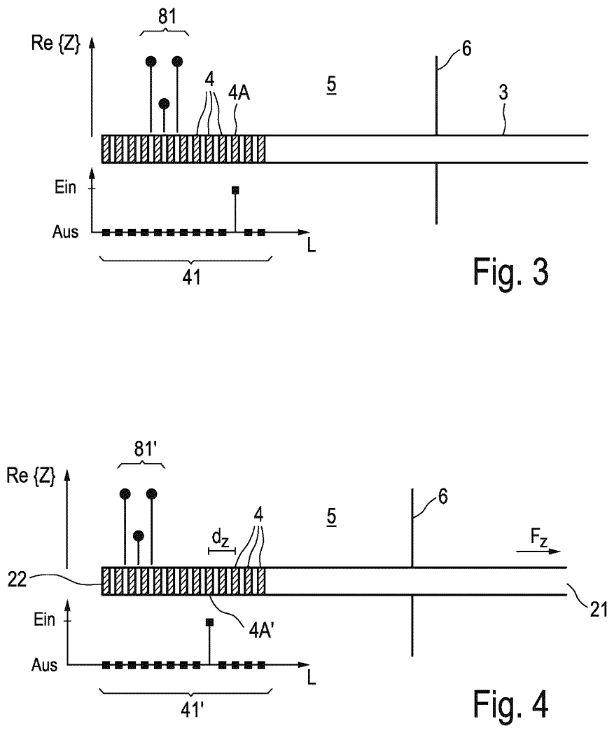

The system 1 makes use of this property of the body tissue 5 for determining an axial dislocation. In the following, a method for adapting the output of electrical stimuli to an axial dislocation is described in the following with reference to FIGS. 3 and 4.

The stimulator shaft 3 is positioned with the distal end section 23 in the vicinity of the nerve which is not shown in any more detail in the present case. The stimulator shaft 3 is surrounded by body tissue 5 at least in the region of the distal end section 23. To generate an electrical stimulus or a plurality of successive electrical stimuli or a continuous electrical stimulus, the control device 7 activates the electrodes 4. An electrode activation pattern 41 is created depending on which electrodes 4 the control device 7 activates and which it deactivates. In the example shown, only the third electrode 4A from last—as viewed in the longitudinal direction L—is activated. In addition, the control device 7 activates all electrodes 4 to output a measuring current. The measuring current is lower than the current of a respective electrode 4 that is output for generating the electrical stimulus. The determining device 8 determines an impedance pattern 81, the real part Re{Z} of which is illustrated in FIG. 3. The impedance pattern 81 is composed of the individual impedances that are determined between the adjacent electrodes 4. As shown, the impedance can take on any desired value between a maximum and a minimum impedance value, depending on a resolution of the determining device 8. The impedance pattern 81 along the longitudinal axis L is characteristic for the position of the stimulator shaft 3 relative to the body tissue 5 and therefore relative to the nerve to be anaesthetized.

If a force F_{Z} then acts on the stimulator shaft 3, this stimulator shaft changes its axial position in relation to the body tissue 5 (axial dislocation). In the present example, the stimulator shaft 3 is pulled out of the body of the patient. As a consequence, the positions of the individual electrodes 4 also change in relation to the body tissue 5, so the electrode activation pattern 41 generated by the control device 7 can no longer achieve the desired pain-reducing effect. In other words, the electrodes 4 no longer form the same electrode activation pattern 41 in relation to the body tissue 5 surrounding the stimulator shaft 3.

After dislocation of the stimulator shaft 3 has taken place, the determining device 8 determines an impedance pattern anew, what is known as a moved impedance pattern 81′. The moved impedance pattern 81′ is moved, with respect to the longitudinal axis L, relatively to the originally determined impedance pattern 81. In the example shown, the moved impedance pattern 81′ is moved compared to the original impedance pattern by the distance d_{Z} in the direction of the distal stimulator end 22. Aside from the movement along the longitudinal axis L, the original impedance pattern 81 and the moved impedance pattern 81′ are identical.

By comparing the original impedance pattern 81 with the moved impedance pattern 81′, the determining device can determine the distance d_{Z}. The distance d_{Z} represents the axial dislocation. In this respect, the distance d_{Z} is equal to the axial dislocation or has at least one only slight deviation from the actual dislocation.

So that the electrode activation pattern 41 can continue to achieve the desired effect in the body tissue 5, it is likewise moved by the distance d_{Z}, actually in the direction of the distal stimulator end 22. For this purpose, the determining device 8 transmits the distance d_{Z} to the control device 7 via the signal line 71. The control device 7 changes the activation of the electrodes 4 depending on the dislocation, i.e. depending on the distance d_{Z}. In the example shown, the control device 7 deactivates the previously activated third electrode 4A from last and instead activates the fifth electrode 4A′ from last. The activation and deactivation of the electrodes 4 that are changed in this way, i.e. newly set up, by the control device forms a moved electrode activation pattern 41′. Aside from the movement along the longitudinal axis L, the original electrode activation pattern 41 and the moved electrode activation pattern 41′ are identical. In other words, the electrode activation pattern 41 reproduces the movement of the impedance pattern 81 along the longitudinal axis L. The electrode activation pattern tracks the impedance pattern.

If the distance d_{Z} is a whole number multiple of the distance between two adjacent electrodes 4, the moved electrode activation pattern 41′ has the same position in relation to the surrounding body tissue 5 as the original electrode activation pattern 41.

In the other case, the control device 7 activates the electrodes 4 to form a moved electrode pattern 41′ in such a manner that the position of the moved electrode activation pattern 41′ has the smallest distance from the position of the original electrode activation pattern 41 with respect to the body tissue 5.

The control device 7 therefore operates such that the electrode activation pattern 41 retains or at least substantially retains its position relative to the body tissue 5 independently or in spite of a dislocation of the stimulator shaft 3. As a result, the electrical stimulus output by means of the electrodes 4 is also the same or at least similar and effects the same or at least a similar effect in the body tissue 5.

1. A system for electrical neurostimulation, the system comprising:
a stimulator that has a stimulator shaft;
a control device; and
a determining device,
the stimulator shaft having a distal stimulator end with electrodes arranged adjacently along a longitudinal axis of the stimulator shaft and configured for outputting electrical stimuli to a body tissue surrounding the stimulator shaft,
the control device connected to the electrodes, the control device configured for controlling an output of the electrical stimuli,
the electrodes configured to be activated and deactivated independently of one another by the control device and activated for forming different electrode activation patterns along the longitudinal axis,
the determining device being configured for determining an axial dislocation of the stimulator shaft,
the control device being connected to the determining device and configured for axially moving an electrode activation pattern by a change of activation and deactivation of the electrodes as a function of the axial dislocation, in order to locally adapt the output of the electrical stimuli to the axial dislocation of the stimulator shaft.

2. The system according to claim 1, wherein the determining device is connected to the electrodes and configured for:
determining impedances between electrodes and therefore an impedance pattern of the body tissue along the longitudinal axis of the stimulator shaft,
determining a temporal change of the impedance pattern, and
determining the axial dislocation depending on the temporal change of the impedance pattern.

3. The system according to claim 2, wherein the determining device is configured for determining impedances between respectively adjacently arranged electrodes.

4. The system according to claim 2, wherein the determining device is configured for determining the temporal change of an impedance spectrum of the impedances.

5. The system according to claim 2, wherein the determining device is configured for determining the temporal change of the impedance pattern, taking account of a real part and/or an imaginary part of the impedances that are determined.

6. The system according to claim 2, wherein the determining device is configured for determining the temporal change of the impedance pattern, taking account of a magnitude and/or a phase of the impedances that are determined.

7. The system according to claim 2, wherein the determining device is configured for determining the temporal change of the impedance pattern, taking account of a spectrum of the impedances that are determined.

8. The system according to claim 2, wherein the electrodes are configured for outputting a measuring current for determination of the impedances, and wherein the control device is configured for controlling the output of the measuring current.

9. The system according to claim 8, wherein the measuring current has a current intensity of at most 10 mA.

10. The system according to claim 8, wherein the measuring current has a current intensity of at most 0.5 mA.

11. The system according to claim 8, wherein the measuring current has a current intensity of at most 0.01 mA.

12. The system according to claim 8, wherein the measuring current is an alternating current which has a frequency of at least 10 Hz.

13. The system according to claim 8, wherein the measuring current is an alternating current which has a frequency of at least 1 kHz.

14. The system according to claim 8, wherein the measuring current is an alternating current which has a frequency spectrum.

15. The system according to claim 14, wherein the alternating current has a sweep.

16. The system according to claim 1, wherein the determining device is configured for determining impedances based on the electrical stimuli.

17. The system according to claim 16, wherein:
the electrodes comprise a first group of electrodes and a second group of electrodes, and
the determining device is configured for determining impedances across the first group of electrodes when an electrical stimulus is output by the second group of electrodes.

18. The system according to claim 17, wherein electrodes of the first group of electrodes are not contained in the second group of electrodes.

19. A method for operating a system for electrical neurostimulation, the system having a stimulator with an elongated stimulator shaft having a distal stimulator end with electrodes, the method comprising the steps of:
activating the electrodes to form an electrode activation pattern with activated and non-activated electrodes;
determining an axial dislocation of the elongated stimulator shaft;
activating the electrodes such that the electrode activation pattern is moved axially depending on the axial dislocation by a changed activation and deactivation of the electrodes, in order to compensate for the axial dislocation of the elongated stimulator shaft.
